(19) United States
(12) Patent Application Publication
(10) Pub. No.: US 2006/0216237 A1
(43) Pub. Date: Sep. 28, 2006

(54) Inhibition of angiogenesis and tumor development by IGFBP-4

(76) Inventors: Peter C. Brooks, Carmel, NY (US); Abebe Akalu, Forest Hills, NY (US); Alexandra Cretu, Wilmington, DE (US); Desiree Policarpio, Douglaston, NY (US)

Correspondence Address: WILSON SONSINI GOODRICH & ROSATI, 650 PAGE MILL ROAD, PALO ALTO, CA, 94304-1050, US

(21) Appl. No.: 11/372,367

(22) Filed: Mar. 9, 2006

Related U.S. Application Data
(60) Provisional application No. 60/660,903, filed on Mar. 11, 2005.

(51) Int. Cl. A61K 51/00; A61K 39/395

(52) U.S. Cl. 424/1.69; 424/145.1

(57) ABSTRACT

The invention provides compositions comprising IGFBP-4 and methods for inhibiting angiogenesis and tumor development processes, and for treating angiogenesis-dependent conditions, using an insulin growth factor binding protein, IGFBP-4.

### RELATED APPLICATIONS

This application claims the benefit of U.S. Provisional Application No. 60/660,903, entitled “INHIBITION OF ANGIOGENESIS AND TUMOR DEVELOPMENT BY IGFBP-4,” filed Mar. 11, 2005, by Peter Brooks et al., which application is incorporated herein by reference in its entirety.

### REFERENCE TO GOVERNMENT GRANT

This invention was made, in part, with government support under NIH RO1 CA91645 awarded by the National Institutes of Health. The government has rights to the invention.

### FIELD OF THE INVENTION

The present invention relates to the field of medicine, specifically to methods and compositions for inhibiting angiogenesis using the insulin growth factor binding protein, IGFBP-4.

### BACKGROUND OF THE INVENTION

Angiogenesis is the physiological process by which new blood vessels develop from pre-existing vessels (Varner, et al., Cell Adh. Commun. 1995, 3:367-374; Blood, et. al., Biochim. Biophys. Acta. 1990, 1032:89-118; Weidner, et al., J. Natl. Cancer Inst. 1992, 84:1875-1887). Angiogenesis has been suggested to play roles in both normal and pathological processes. For example, angiogenic processes are involved in the development of the vascular systems of animal organs and tissues. They are also involved in transitory phases of angiogenesis, for example during the menstrual cycle, in pregnancy, and in wound healing. On the other hand, a number of diseases are known to be associated with deregulated angiogenesis.

In certain pathological conditions, angiogenesis is recruited as a means to provide adequate blood and nutrient supply to the cells within the affected tissue. Many of these pathological conditions involve aberrant cell proliferation or regulation. Therefore, inhibition of angiogenesis is a potentially useful approach to treating diseases that are characterized by unregulated blood vessel development. For example, angiogenesis is involved in pathologic conditions including ocular diseases, e.g., macular degeneration, neovascular glaucoma, retinopathy of prematurity, and diabetic retinopathy; inflammatory diseases, e.g., immune and non-immune inflammation, rheumatoid arthritis, osteoarthritis, chronic articular rheumatism and psoriasis; chronic inflammatory diseases, e.g. ulcerative colitis and Crohn's disease; corneal graft rejection; vitamin A deficiency; Sjorgen's disease; acne rosacea; mycobacterium infections; bacterial and fungal ulcers; Herpes simplex infections; systemic lupus; retrolental fibroplasia; rubeosis; capillary proliferation in atherosclerotic plaques, and; osteoporosis. Angiogenesis is also involved in cancer-associated disorders, including, for example, solid tumors, tumor metastases, blood borne tumors such as leukemias, angiofibromas, Kaposi's sarcoma, benign tumors such as hemangiomas, acoustic neuromas, neurofibromas, trachomas, and pyogenic granulomas, as well as other cancers which require neovascularization to support tumor growth. Other angiogenesis-dependent conditions include, for example, hereditary diseases such as Osler-Weber Rendu disease and haemorrhagic teleangiectasia; myocardial angiogenesis; plaque neovascularization; hemophiliac joints and wound granulation. Progression of tumors such as melanoma, from benign to metastatic disease, correlates with an increase in angiogenesis as well as an increase in expression of specific cell adhesion receptors including integrins (Srivastava, et al., Am. J. Pathol. 1988, 133:419-423; Koth, et al., N. Engl. J. Med. 1991, 325:171-182). Thus, angiogenesis likely plays a critical role in melanoma progression.

Examples of normal physiological processes involving angiogenesis include embryo implantation, embryogenesis and development, and wound healing. It is conceivable that angiogenesis can also be altered to beneficially influence normal physiological processes. Furthermore, studies have indicated that adipose tissue growth is dependent on angiogenesis, likely due to the need for recruitment of new blood vessels. Delivery of an angiogenesis inhibitor to mice was found to reduce diet-induced obesity, the most common type of obesity in humans (Brakenhielm, et al., Circ. Res. 2004, 94 (12):1579-88). This finding suggests a utility for angiogenesis inhibitors in addressing obesity and certain related conditions. Therefore, the inhibition of angiogenesis potentially can be applied in normal angiogenic responses where a prophylactic or therapeutic need or benefit exists.

Angiogenesis involves the degradation of components of the extracellular matrix and then the migration, proliferation and differentiation of endothelial cells to form tubules and eventually new vessels. It requires cooperation of a variety of molecules including growth factors, cell adhesion receptors, matrix degrading enzymes and extracellular matrix components (Varner, et al., Cell Adh. Commun. 1995, 3:367-374; Blood, et. al., Biochim. Biophys. Acta. 1990, 1032:89-118; Weidner, et al., J. Natl. Cancer Inst. 1992, 84:1875-1887).

Studies have suggested that angiogenesis requires proteolytic remodeling of the extracellular matrix (ECM) surrounding blood vessels in order to provide a microenvironment conducive to new blood vessel development (Varner, et al., Cell Adh. Commun. 1995, 3:367-374; Blood, et. al., Biochim. Biophys. Acta. 1990, 1032:89-118; Weidner, et al., J. Natl. Cancer Inst. 1992, 84:1875-1887; Weidner, N. et al., N. Engl. J. Med. 1991; 324:1-7; Brooks, P. C. et al. J. Clin. Invest. 1995; 96:1815-1822; Brooks, P. C. et al., Cell 1994; 79:1157-1164). The extracellular matrix protein, collagen, makes up over 25% of the total protein mass in animals and the majority of protein within the ECM. Proteolytic exposure of unique matrix immobilized cryptic epitopes and subsequent cellular interactions with these epitopes, which serve as regulatory sites, play crucial roles in angiogenesis, tumor growth and metastasis.

Extracellular matrix components include, e.g., collagen, fibronectin, osteopontin, laminin, fibrinogen, elastin, thrombospondin, tenascin, and vitronectin. Studies have identified cryptic sites, including HUIV26, within the collagen, that regulates angiogenesis and endothelial cell behavior (Xu, et al., Hybridoma 2000, 19:375-385; Xu, et al., J. Cell Biol 2001, 154:1069-1079; Hangai, et al., Am. J. Pathol 2002, 161:1429-1437; Lobov, et al., Proc. Natl. Acad. Sci USA 2002, 99:11205-11210). This functional cryptic site was shown to be highly expressed within the ECM of malignant tumors and within the sub-endothelial basement membrane of tumor-associated blood vessels, and its exposure found to be involved in the regulation of angiogenesis in vivo (Xu, et al., Hybridoma 2000, 19:375-385; Xu, et al., J. Cell Biol 2001, 154:1069-1079; Hangai, et al., Am. J. Pathol 2002, 161:1429-1437; Lobov, et al., Proc. Natl. Acad. Sci USA 2002, 99:11205-11210, and U.S. Ser. No. 09/478,977, now U.S. Pub. No. 2003/0113331, the disclosure of which is incorporated herein by reference in its entirety).

Studies have demonstrated that the HUIV26 cryptic epitope is specifically exposed within collagen type-IV of tumors and angiogenic blood vessels. Moreover, a function-blocking monoclonal antibody specifically directed to the HUIV26 cryptic site potently inhibits angiogenesis, tumor growth and metastasis in several in vivo models. Therefore, the possibility exists that cellular (tumor, stromal and endothelial cell) interactions with unique cryptic ECM sites may specifically modulate signaling pathways involved in controlling invasive cellular behavior, including angiogenesis, tumor growth and metastasis.

Molecular alterations that occur in both tumor and stromal cells are thought to potentiate angiogenesis in part by modifying expression and bioavailability of angiogenic growth factors as well as altering expression of matrix-degrading proteases. Collectively, these and other molecular changes help to create a microenvironment conducive to new blood vessel growth, one factor that contributes to metastasis and tumor growth. There is evidence for the importance of numerous molecular regulators that contribute to new blood vessel growth, including matrix-degrading proteases such as MMP-9, angiogenesis inhibitors such as TSP-1 and angiogenic growth factors such as VEGF (see, e.g., Yu, et al., Proc. Natl. Acad. Sci. USA 1999, 96:14517-14522 and Dameron, et al., Science 1994, 265:1582-1584). These molecular regulators, the proteins that in turn regulate them, and any of a number of other molecules potentially affect angiogenesis and metastasis. However, the exact mechanisms of the regulation of these and related processes, including the genes and gene expression patterns involved, have not been determined

Proteolytic activity plays a crucial role in controlling angiogenesis by releasing matrix-sequestered growth factors as well as remodeling ECM proteins. ECM remodeling results in the exposure of cryptic epitopes, such as the HUIV26 collagen site and sites within laminin. The HUIV26 cryptic collagen epitope is recognized by αvβ3 integrin, which is expressed in tumors.

Certain proteins appear to be involved in integrin signaling, for example, Insulin Growth Factor Binding Proteins (IGFBPs). IGFBPs are a family of secreted proteins that function to regulate IGF-signaling by binding to IGFs, thereby disrupting IGF receptor binding and subsequent signaling (Pollak, et al., Nat. Rev. Cancer 2004, 4:505-518; Mohan, et al., J. Endocrinol. 2002, 175:19-31; LeRoith, et al., Cancer Lett. 2003, 195:127-137). Specific IGFBPs might directly bind to integrin receptors, thereby modulating their function independently from IGFs (McCaig, et al., J. Cell Sci. 2002, 115:4293-4303; Schutt, et al., J. Mol. Endocrinol. 2004, 32:859-868; Furstenberger, et al., Lancet. 2002, 3:298-302). Therefore, IGFBPs might regulate angiogenesis, cellular adhesion, migration and tumor growth by both IGF-dependent and independent mechanisms (McCaig, et al., J. Cell Sci. 2002, 115:4293-4303; Schutt, et al., J. Mol. Endocrinol. 2004, 32:859-868; Furstenberger, et al., Lancet. 2002, 3:298-302) (Mazerbourg, et al., Growth Horm. IGF. Res. 2004, 14:71-84). However, in vivo regulation of these cellular processes, including angiogenesis, by integrin-receptor binding of IGFBPs has not been established previously.

### SUMMARY OF THE INVENTION

The present invention relates to the discovery that IGFBP-4 is an inhibitor of angiogenesis. In embodiments, the invention provides methods for the inhibiting angiogenesis in a tissue, thereby inhibiting events in the tissue which depend upon angiogenesis.

Specifically, the invention relates to therapeutic compositions comprising IGFBP-4 and a pharmaceutically acceptable excipient. The invention also relates to methods for inhibiting angiogenesis, treating a tumor, inhibiting metastasis, or treating an angiogenesis-dependent condition in a patient comprising: administering a therapeutically effective amount of IGFBP-4 to the patient.

In embodiments of these methods, the IGFBP-4 is administered: intravenously, intraperitoneally, intramuscularly, subcutaneously, intracavity, transdermally, topically, intraocularly, orally, intranasally, or by peristaltic means. In related embodiments the IGFBP-4 is administered in combination with another compound, e.g., an inhibitor of angiogenesis and tumor development processes (e.g., a monoclonal antibody that binds to the cryptic collagen epitope, HUIV26), a chemotherapeutic agent, a radioactive material, or in conjunction with a cytostatic agent. Embodiments also include methods in which the patient is a mammal, and in specific embodiments, the patient is a human.

### BRIEF DESCRIPTION OF THE DRAWINGS

FIG. 1 Inhibition of αvβ3-Mediated Ligation of the HUIV26 Cryptic Collagen Epitope Increases IGFBP-4 Expression. As described in Example I, incubation of cells with Mab HUIV26, as compared to isotype-matched controls, resulted in an approximately 115-fold increase in the relative levels of IGFBP-4 RNA.

FIG. 2 Isolation of αvβ3 Expression Variants of Human ECV Bladder Carcinoma (Parental Cells). Human ECV304 carcinoma cells were subjected to FACS following incubation with Mab LM609 (anti-αvβ3). Four negative selections for expression of αvβ3 integrin were carried out. The figure shows a histogram of FACS analysis for surface expression of integrins αvβ3 (Mab LM609), β1 (Mab P4C10) or control (non-specific Ab) in parental carcinoma cells (ECV).

FIG. 3 Isolation of αvβ3 Expression Variants of Human ECV Bladder Carcinoma (Variant Cells). Human ECV304 carcinoma cells were subjected to FACS following incubation with Mab LM609 (anti-αvβ3). Four negative selections for expression of αvβ3 integrin were carried out. The figure shows a histogram of FACS analysis for surface expression of integrins αvβ3 (Mab LM609), β1 (Mab P4C10) or control (non-specific Ab) in negative selected carcinoma cells (ECVL).

FIG. 4 Elevated Levels of IGFBP-4 in CM from Tumor Cells Lacking αvβ3 as Demonstrated by ELISA. Conditioned Medium (CM) was evaluated for the relative levels of IGFBP-4 by ELISA. The figure shows data obtained using CM (25 μl), from ECV and ECVL tumor cells, diluted in coating buffer 1:1 and incubated in microtiter wells. The wells were washed, blocked and incubated with anti-IGFP-3 and IGFBP-4 Mabs. The relative levels of IGFBP-3 and IGFBP-4 were detected by incubation with HRP-labeled goat anti-mouse antibody. All data were corrected for non-specific binding. Data bars represent the mean O.D±standard deviations from triplicate wells. The relative levels of IGFBP-4 increased in CM from ECVL by greater than 10-fold as compared to ECV, while little if any change in the levels of IGFBP-3 was observed. Experiments were completed 3 times with similar results.

FIG. 5 Elevated Levels of IGFBP-4 in CM from Tumor Cells Lacking αvβ3 as Demonstrated by Western Blotting. CM was examined for the relative levels of IGFBP-4 by Western blot analysis. The figure shows analysis of CM from ECV and ECVL cells, for IGFBP-4, or using soluble fibronectin as control. IGFBP-4 was dramatically increased in the CM of ECVL cells as compared to ECV cells while little or no change was detected in soluble fibronectin.

FIG. 6 Recombinant IGFBP-4 Inhibits Angiogenesis. Filter discs with basic fibroblast growth factor (bFGF) (12 ng) were placed on the CAMs of 10-day old chick embryos. Twenty-four hours later the embryos were treated topically with 100 ng of IGFBP-4 of BSA as a control. At the end of 3 days the CAMs were removed and angiogenesis quantified by counting blood vessel branch points. Data bars represent the mean branch points±standard deviation from 8 to 10 embryos per condition. The Angiogenic Index=mean branch points from each condition minus the mean branch points in absence of bFGF. IGFBP-4 (100 ng) significantly (P<0.001) inhibited bFGF-induced angiogenesis by greater than 70% as compared to control. Experiments were completed twice with similar results.

FIG. 7 Recombinant IGFBP-4 Inhibits Tumor Cell Adhesion to Denatured Collagen Type-IV. Culture plates (48-well) were coated with either vitronectin (5.0 μg/ml) or denatured collagen (10.0 μg/ml). M21 tumor cells were incubated in adhesion buffer in the presence or absence of IGFBP-4 or control BSA (200 ng/ml) for 1 hour at 37° C. then added to the ECM-coated wells. Cells were allowed to attach for 20 minutes. Attached cells were stained with crystal violet. Cell adhesion was quantified by measuring the optical density (O.D.) of eluted dye at 560 nm. Data bars represent mean O.D±standard deviations from triplicate wells. IGFBP-4 potently inhibited M21 cell adhesion to denatured collagen type-IV by approximately 70% while exhibiting little effect on adhesion to vitronectin. Experiments were completed twice with similar results.

FIG. 8 Inhibition of αvβ3-Mediated Ligation Increases IGFBP-4 RNA Expression in M21 Cells. M21 cells were seeded on denatured collagen type-IV coated plates in the presence or absence of anti-αvβ3 specific Mab LM609 or an isotype-matched control antibody. Cells were allowed to incubate for 12 hours in 1.0% serum-containing medium. Expression of IGFBP-4 was examined by RT-PCR. Expression levels were normalized for β2 macroglobulin (B2M). The relative level of IGFBP-4 was elevated by approximately 8-fold in M21 cells treated with the anti-αvβ3 specific Mab LM609 as compared to an isotype-matched control antibody.

FIG. 9 Inhibition of αvβ3-Mediated Ligation Increases IGFBP-4 RNA Expression in M21 Tumors. M21 cells were seeded on denatured collagen type-IV coated plates in the presence or absence of anti-αvβ3 specific Mab LM609 or an isotype matched control antibody. Cells were allowed to incubate for 12 hours in 1.0% serum-containing medium. The figure shows expression of IGFBP-4 in M21 tumors grown in chick embryo from either untreated (NT) or treated systemically with Mab LM609 or control non-specific antibody (100 μg/embryo) N=5. Expression of IGFBP-4 was significantly enhanced in M21 tumors grown in the chick embryo following treatment with Mab LM609.

### DETAILED DESCRIPTION OF THE INVENTION

In describing the present invention, the following terms will be employed, and are intended to be defined as indicated below. Unless otherwise indicated, all terms used herein have the same ordinary meaning as they would to one skilled in the art of the present invention.

Citation of documents herein is not intended as an admission that any of the documents cited herein is pertinent prior art, or an admission that the cited documents are considered material to the patentability of the claims of the present application. All statements as to the date or representations as to the contents of these documents are based on the information available to the applicant and do not constitute any admission as to the correctness of the dates or contents of these documents.

### Definitions

I. Angiogenesis and Diseases Potentially Treated by Inhibitors of Angiogenesis

As used herein, the terms “angiogenesis inhibitory,” “angiogenesis inhibiting” or “anti-angiogenic” include vasculogenesis, and are intended to mean effecting a decrease in the extent, amount, or rate of neovascularization. Effecting a decrease in the extent, amount, or rate of endothelial cell proliferation or migration in the tissue is a specific example of inhibiting angiogenesis.

The term “angiogenesis inhibitory composition” refers to a composition which inhibits angiogenesis-mediated processes such as endothelial cell migration, proliferation, tube formation and subsequently leading to the inhibition of the generation of new blood vessels from existing ones, and consequently affects angiogenesis-dependent conditions.

As used herein, the term “angiogenesis-dependent condition” is intended to mean a condition where the process of angiogenesis or vasculogenesis sustains or augments a pathological condition, or beneficially influence normal physiological processes. Angiogenesis is the formation of new blood vessels from pre-existing capillaries or post-capillary venules. Vasculogenesis results from the formation of new blood vessels arising from angioblasts which are endothelial cell precursors. Both processes result in new blood vessel formation and are included in the meaning of the term angiogenesis-dependent conditions. Similarly, the term “angiogenesis” as used herein is intended to include de novo formation of vessels such as those arising from vasculogenesis as well as those arising from branching and sprouting of existing vessels, capillaries and venules.

Examples of diseases in which angiogenesis plays a role in the maintenance or progression of the pathological state are listed herein in the Background of the Invention. Additional diseases are known to those skilled in the art and are similarly intended to be included within the meaning of “angiogenesis-dependent condition” and similar terms as used herein.

II. Cancers, Tumors and Tissues

The methods of the invention are contemplated for use in treatment of a tumor tissue of a patient with a tumor, solid tumor, a metastasis, a cancer, a melanoma, a skin cancer, a breast cancer, a hemangioma or angiofibroma and the like cancer, and the angiogenesis to be inhibited is tumor tissue angiogenesis where there is neovascularization of a tumor tissue. Typical solid tumor tissues treatable by the present methods include, but are not limited to, tumors of the skin, melanoma, lung, pancreas, breast, colon, laryngeal, ovarian, prostate, colorectal, head, neck, testicular, lymphoid, marrow, bone, sarcoma, renal, sweat gland, and the like tissues. Further examples of cancers treated are glioblastomas.

A tissue to be treated is a retinal tissue of a patient with diabetic retinopathy, macular degeneration or neovascular glaucoma and the angiogenesis to be inhibited is retinal tissue angiogenesis where there is neovascularization of retinal tissue.

Thus, methods which inhibit angiogenesis in a diseased tissue ameliorate symptoms of the disease and, depending upon the disease, can contribute to cure of the disease. In embodiments, the invention contemplates inhibition of angiogenesis in a tissue. The extent of angiogenesis in a tissue, and therefore the extent of inhibition achieved by the present methods, can be evaluated by a variety of methods, such as are described herein.

Any of a variety of tissues, or organs comprised of organized tissues, can support angiogenesis in disease conditions including skin, muscle, gut, connective tissue, joints, bones and the like tissue in which blood vessels can invade upon angiogenic stimuli. Thus, in one embodiment, a tissue to be treated is an inflamed tissue and the angiogenesis to be inhibited is inflamed tissue angiogenesis where there is neovascularization of inflamed tissue. In this class the method contemplates inhibition of angiogenesis in arthritic tissues, such as in a patient with chronic articular rheumatism, in immune or non-immune inflamed tissues, in psoriatic tissue and the like.

In the absence of neovascularization of tumor tissue, the tumor tissue does not obtain the required nutrients, slows in growth, ceases additional growth, regresses and ultimately becomes necrotic resulting in killing of the tumor. The present invention provides for a method of inhibiting tumor neovascularization by inhibiting tumor angiogenesis according to the present methods. Similarly, the invention provides a method of inhibiting tumor growth by practicing the angiogenesis-inhibiting methods.

The methods are also particularly effective against the formation of metastases because their formation requires vascularization of a primary tumor so that the metastatic cancer cells can exit the primary tumor and their establishment in a secondary site requires neovascularization to support growth of the metastases.

The invention also contemplates the practice of the method in conjunction with other therapies such as conventional chemotherapy directed against solid tumors and for control of establishment of metastases. The administration of an angiogenesis inhibitor is typically conducted during or after chemotherapy, although it is preferable to inhibit angiogenesis after a regimen of chemotherapy at times where the tumor tissue will be responding to the toxic assault by inducing angiogenesis to recover by the provision of a blood supply and nutrients to the tumor tissue. In addition, it is preferred to administer the angiogenesis inhibition methods after surgery where solid tumors have been removed as a prophylaxis against metastases.

III. Patients

The patient treated in the present invention in its many embodiments is desirably a human patient, although it is to be understood that the principles of the invention indicate that the invention is effective with respect to all mammals, which are intended to be included in the term “patient.” In this context, a mammal is understood to include any mammalian species in which treatment of diseases associated with angiogenesis is desirable, particularly agricultural and domestic mammalian species.

IV. IGFBPs

IGFBPs have been described in e.g., Pollak, et al., Nat. Rev. Cancer 2004, 4:505-518; Mohan, et al., J. Endocrinol. 2002, 175:19-31; and LeRoith, et al., Cancer Lett. 2003, 195:127-137. It is conceivable that IGFBPs administered according to the methods of the invention might directly bind to integrin receptors, thereby modulating their function independently from IGFs (McCaig, et al., J. Cell Sci. 2002, 115:4293-4303; Schutt, et al., J. Mol. Endocrinol. 2004, 32:859-868; Furstenberger, et al., Lancet. 2002, 3:298-302). Therefore, IGFBPs might regulate angiogenesis, cellular adhesion, migration and tumor growth by both IGF-dependent and independent mechanisms (McCaig, et al., J. Cell Sci. 2002, 115:4293-4303; Schutt, et al., J. Mol. Endocrinol. 2004, 32:859-868; Furstenberger, et al., Lancet. 2002, 3:298-302; Mazerbourg, et al., Growth Horm. IGF. Res. 2004, 14:71-84; and Mazerbourg, et al., Growth Horm. IGF. Res. 2004, 14:71-84.

Human IGFBP-1, IGFBP-2, IGFBP-3, IGFBP-4, IGFBP-5, IGFBP-6, IGFBP-7, IGFBP-8, IGFBP-9, and IGFBP-10, are examples of known proteins that belong to the IGFBP superfamily and are registered in the protein amino acid database SWISSPROT or the nucleotide sequence database GenBank.

As described in U.S. Publication No. 2004/0072238, incorporated herein by reference, six types of molecules, IGFBP-1 to 6, among the IGFBP superfamily have structural similarity and reportedly bind with higher affinity to IGF than to insulin. Therefore, they are classified into a subfamily as high-IGF-affinity IGFBPs (Mol. Endocrinol. 1988, 2:404; EMBO J. 1999, 8, 2497; Mol. Endocrinol. 1989, 2:1176; Mol. Endocrinol. 1990, 4:1806; Biochem. Biophys. Res. Commun. 1991, 176: 219; J. Biol. Chem. 1991, 266: 9043; J. Biol. Chem. 1991, 266: 10646).

The invention contemplates the administration of IGFBP-4 protein in recombinant or purified form, or provided as part of a nucleic acid construct (e.g., “naked DNA”) by methods known to those of skill in the art. In further embodiments, as described further below, a cleavage product of IGFBP-4 is used in the methods of the invention.

### MODES OF CARRYING OUT THE INVENTION

I. Angiogenesis Assays

Angiogenesis assays useful in the methods of the invention are described in e.g., U.S. Ser. No. 09/478,977 (U.S. Pub. No. 2003/0113331), U.S. Publication No. 2004/242490 A1, and WO 2004/073649. The disclosures of these publications are incorporated herein by reference in their entirety.

Methods of measuring alterations in angiogenesis are well known in the art. For example, angiogenesis can be measured in the chick chorioallantoic membrane (CAM). This assay is referred to as the CAM assay. The CAM assay has been described in detail by others, and further has been used to measure both angiogenesis and neovascularization of tumor tissues. See Ausprunk et al., Am. J. Pathol. 1975, 79:597-618 and Ossonski et al., Cancer Res., 1980, 40:2300-2309). The CAM assay is a well-recognized assay model for in vivo angiogenesis because neovascularization of whole tissue is occurring, and actual chick embryo blood vessels are growing into the CAM or into the tissue grown on the CAM.

The CAM assay is particularly useful because there is an internal control for toxicity in the assay system. The chick embryo is exposed to any test reagent, and therefore the health of the embryo is an indication of toxicity.

Alterations in angiogenesis can also be measured using the in vivo rabbit eye model, referred to as the rabbit eye assay. The rabbit eye assay has been described in detail by others, and further has been used to measure both angiogenesis and neovascularization in the presence of angiogenic inhibitors such as thalidomide. See D'Amato et al. Proc. Natl. Acad. Sci. 1994, 91:4082-4085.

The rabbit eye assay is a well recognized assay model for in vivo angiogenesis because the neovascularization process, exemplified by rabbit blood vessels growing from the rim of the cornea into the cornea, is easily visualized through the naturally transparent cornea of the eye. Additionally, both the extent and the amount of stimulation or inhibition of neovascularization or regression of neovascularization can easily be monitored over time. Finally, the rabbit is exposed to any test reagent, and therefore the health of the rabbit is an indication of toxicity of the test reagent.

Another assay measures angiogenesis in the chimeric mouse:human mouse model and is referred to as the chimeric mouse assay. This assay is described herein, and in detail by others, as a method for measuring angiogenesis, neovascularization, and regression of tumor tissues. See Yan, et al. (1993) J. Clin. Invest. 91:986-996.

The chimeric mouse assay is a useful assay model for in vivo angiogenesis because the transplanted skin grafts closely resemble normal human skin histologically, and neovascularization of whole tissue is occurring wherein actual human blood vessels are growing from the grafted human skin into the human tumor tissue on the surface of the grafted human skin. The origin of the neovascularization into the human graft can be demonstrated by immunohistochemical staining of the neovasculature with human-specific endothelial cell markers.

The chimeric mouse assay demonstrates regression of neovascularization based on both the amount and extent of regression of new vessel growth. Furthermore, it is easy to monitor effects on the growth of any tissue transplanted upon the grafted skin, such as a tumor tissue. Finally, the assay is useful because there is an internal control for toxicity in the assay system. The chimeric mouse is exposed to any test reagent, and therefore the health of the mouse is an indication of toxicity.

To confirm the effects of a compound, e.g., IGFBP-4, on angiogenesis, the mouse Matrigel plug angiogenesis assay can be used. Various growth factors (IGF-1, bFGF or VEGF) (250 ng) and Heparin (0.0025 units per/ml) are mixed with growth factor reduced Matrigel as previously described (Montesano, et al., J. Cell Biol. 1983, 97: 1648-1652; Stefansson, et al., J. Biol. Chem. 2000, 276: 8135-8141). IGFBP-4 or control BSA (10 to 500 ng) can be included in the Matrigel preparations. In control experiments, Matrigel is prepared in the absence of growth factors. Mice are injected subcutaneously with 0.5 ml of the Matrigel preparation and allowed to incubate for one week. Following the incubation period, the mice are sacrificed and the polymerized Matrigel plugs surgically removed. Angiogenesis within the Matrigel plugs is quantified by two established methods, including immunohistochemical analysis and hemoglobin content (Furstenberger, et al., Lancet. 2002, 3: 298-302; Volpert, et al., Cancer Cell 2002, 2(6): 473-83; Su, et al., Cancer Res. 2003, 63: 3585-3592). For immunohistochemical analysis, the Matrigel plugs are embedded in OCT, snap frozen and 4 μm sections prepared. Frozen sections are fixed in methanol/acetone (1:1). Frozen sections are stained with polyclonal antibody directed to CD31. Angiogenesis is quantified by microvascular density counts within 20 high powered (200×) microscopic fields.

Hemoglobin content can be quantified as described previously (Schnaper, et al., J. Cell Physiol. 1993, 256: 235-246; Montesano, et al., J. Cell Biol. 1983, 97: 1648-1652; Stefansson, et al., J. Biol. Chem. 2000, 276: 8135-8141; Gigli, et al., J. Immunol. 1986, 100: 1154-1164). The Matrigel implants are snap frozen on dry ice and lyophilized overnight. The dried implants are resuspended in 0.4 ml of 1.0% saponin (Calbiochem) for one hour, and disrupted by vigorous pipetting. The preparations are centrifuged at 14,000 g for 15 minutes to remove any particulates. The concentration of hemoglobin in the supernatant is then determined directly by measuring the absorbency at 405 nm and compared to a standard concentration of purified hemoglobin. This method of quantification has been used successfully and has been shown to correlate with angiogenesis (Schnaper, et al., J. Cell Physiol. 1993, 256: 235-246; Montesano, et al., J. Cell Biol. 1983, 97: 1648-1652; Stefansson, et al., J. Biol. Chem. 2000, 276: 8135-8141; Gigli, et al., J. Immunol. 1986, 100: 1154-1164).

II. Methods of Assaying Cell Adhesion

Cell adhesion can be measured by methods known to those of skill in the art. Assays have been described previously, e.g. by Brooks, et al., J. Clin. Invest 1997, 99:1390-1398. The Examples describe one such in vitro cell adhesion assay, in which cells are allowed to adhere to substrate (i.e., denatured collagen type-IV) on coated wells, non-attached cells are removed by washing, and non-specific binding sites are blocked by incubation with BSA. The attached cells are stained with crystal violet, and cell adhesion is quantified by measuring the optical density of eluted crystal violet from attached cells at a wavelength of 600 nm.

Specifically, the effects of IGFBP-4 on cell adhesion can be evaluated as follows: non-tissue culture 48-well plates are coated with either intact or proteolyzed ECM proteins including collagen types-I and IV, and fibronectin. To prepared proteolyzed collagen, the wells are first coated with triple helical collagen-I and IV, followed by incubation for 2 hours with activated MMP-1 or MMP-9 (Chemicon International). In control experiments, latent inactive forms of MMP-1 and MMP-9 can be used. The cleavage is stopped after two hours by addition of the MMP inhibitors EDTA and NEM, and the plates washed extensively with PBS. Similar procedures can be followed to cleave fibronectin by incubation with the serine protease plasmin followed by incubation with serine protease inhibitor (aprotinin) to stop the proteolysis. Human endothelial cells (HUVECs or MVEC) (1×10⁵) or melanoma cells (M21 and G361) can be pre-incubated in the presence or absence of IGFBP-4 or control BSA (10 to 500 ng/ml) for 30 to 60 minutes in adhesion buffer, then allowed to attach in the presence or absence of IGFBP-4 or the non-specific control protein BSA (10 to 500 ng/ml). Non-bound cells are removed by washing and attached cells are stained with crystal violet. The wells are next washed and the cell-associated crystal violet eluted with 10% acetic acid as described previously (Petitclerc, et al., J. Biol. Chem. 2000, 275: 8051-8061). Cell adhesion is quantified by measuring the optical density of eluted crystal violet at 600 nm.

III. Methods of Assaying Cell Migration

Assays for cell migration have been described in the literature, e.g., by Brooks, et al., J. Clin. Invest 1997, 99:1390-1398 and methods for measuring cell migration are known to those of skill in the art. In one method for measuring cell migration described herein in the Examples, membranes from transwell migration chambers are coated with substrate (here, thermally denatured collagen), the transwells washed, and non-specific binding sites blocked with BSA. Tumor cells from sub-confluent cultures are harvested, washed, and resuspended in migration buffer in the presence or absence of assay antibodies. After the tumor cells are allowed to migrate to the underside of the coated transwell membranes, the cells remaining on the top-side of the membrane are removed and cells that migrate to the under-side are stained with crystal violet. Cell migration is then quantified by direct cell counts per microscopic field.

Specifically, modulation of endothelial and tumor cell migration on specific ECM proteins by IGFBP-4 can be studied as follows: membranes (8.0 μm pore size) from transwell migration chambers are prepared with either intact or proteolyzed collagen-I and IV, or fibronectin. Endothelial and melanoma cells are pre-incubated in the presence or absence of IGFBP-4 or control BSA (10 to 500 ng/ml) for 30 to 60 minutes in migration buffer then added to the upper chamber of the transwells in the presence or absence of IGFBP-4 or the non-specific control protein BSA (10 to 500 ng/ml) and allowed to migrate for 6 to 12 hours. Following the migration period, the cells on the top-side are removed and the cells that have migrated to the under side are stained with crystal violet. Cell migration is quantified by measuring the optical density of eluted dye with a microtiter plate reader.

IV. Methods of Assaying Tumor Growth

Tumor growth can be assayed by methods known to those of skill in the art, e.g., as described in (Xu, et al., J. Cell Biol 2001, 154:1069-1079). An assay for chick embryo tumor growth can be performed as follows: Single cell suspensions of CS1 melanoma (5×10⁶ per embryo) or HT1080 fibrosarcoma (4×10⁵ per embryo) are applied in a total volume of 40 μl of RPMI to the CAMs of 10-day-old embryos (Brooks et al., 1998). Twenty four hours later, the embryos receive a single intravenous injection of purified Mab HUIV26 or control Mab (100 μg per embryo). Tumors are grown for 7 days, then resected and wet weights are determined. Experiments can be performed with five to ten embryos per condition.

Another method for assaying tumor growth makes use of the SCID mouse, as follows:

Subconfluent human M21 melanoma cells are harvested, washed, and resuspended in sterile PBS (20×10⁶ per ml). SCID mice are injected subcutaneously with 100 μl of M21 human melanoma cell (2×10⁶) suspension. Three days after tumor cell injection, mice are either untreated or treated intraperitoneally (100 μg/mouse) with either Mab HUIV26 or an isotype-matched control antibody. The mice are treated daily for 24 days. Tumor size is measured with calipers and the volume estimated using the formula V×L2×W/2, where V is equal to the volume, L is equal to the length, and W is equal to the width.

Malignant melanoma growth and angiogenesis within a true human tissue microenvironment where cutaneous melanoma typically arise, can be studied as follows: full thickness human neonatal foreskin is surgically transplanted on the backs of nude mice as described (Yan, et al., J. Clin. Invest. 91:986-996. (1993); Petitclerc, et al., Cancer Res. 1999, 59:2724-2730; Berking, et al., Cancer. Res. 2004, 64: 807-811). The grafts are allowed to heal for approximately 3 to 4 weeks at which time a single cell suspension of human M21 or G361 melanoma cells are injected intradermally within the human skin. Tumors are allowed to grow for approximately 2 weeks until they reach a mean tumor size of approximately 100 mm³. The mice are untreated or injected with either IGFBP-4 or control BSA (10 to 500 μg/mouse) 3 times per week. Tumor volumes are estimated using the formula V=L2×W/2 where V=volume, L=length and W=width. At least 14 mice are used per experimental condition to allow 90% power when the differences between the mean tumor sizes is 1.25 standard deviations less than controls. At the end of the tumor growth assay, the mice are sacrificed and the resulting tumors resected and wet weights determined. In addition, tumors are embedded, snap frozen and tissue sections prepared. Tumor-associated angiogenesis is quantified by microvascular density counts as described above. To gain molecular insight into potential mechanisms to account for any anti-tumor activity, frozen tumor sections are fixed and incubated with 0.1% TX-100 for 5 minutes. The tissues are washed and blocked with 2.5% BSA in PBS. Tissue sections can be stained for Ki67 and ApopTag by immunofluoresence. To quantify the relative levels of the indicated proteins at least ten 200× microscopic fields can be assessed from at least 3 tumors from each experimental condition. The total FITC signal is digitized and pixel density for each area quantified by laser confocal image analysis as described previously (Brooks, et al., Science 1994, 264:569-571). In control experiments, tissue sections are incubated with isotype-matched nonspecific antibodies as well as FITC-labeled secondary antibody only.

V. Preparation of IGFBP-4

Methods of producing and preparing proteins for patient administration have been described and are well known in the art.

For example, recombinantly-produced proteins of the present invention can be directly expressed or expressed as fusion proteins. The recombinant protein can be purified by a combination of cell lysis (e.g., sonication, French press) and affinity chromatography. For fusion products, subsequent digestion of the fusion protein with an appropriate proteolytic enzyme can release the desired recombinant protein.

Polynucleotides may be cloned, using standard cloning and screening techniques, from a cDNA library, (see for instance, Sambrook et al., Molecular Cloning: A Laboratory Manual, 2nd Ed., Cold Spring Harbor Laboratory Press, Cold Spring Harbor, N.Y. (1989)). These polynucleotides can also be obtained from natural sources such as genomic DNA libraries or can be synthesized using well known and commercially available techniques.

The polynucleotide including the gene sequence may include the coding sequence for the mature polypeptide, by itself, or the coding sequence for the mature polypeptide in reading frame with other coding sequences, such as those encoding a leader or secretory sequence, a pre-, or pro- or prepro-protein sequence, or other fusion peptide portions. For example, a marker sequence that facilitates purification of the fused polypeptide can be encoded. Polynucleotides can also contain non-coding 5′ and 3′ sequences, such as transcribed, non-translated sequences, splicing and polyadenylation signals, ribosome binding sites and sequences that stabilize mRNA.

There are a number of methods available and well known to those skilled in the art to obtain full-length cDNAs, or extend short cDNAs, for example those based on the method of Rapid Amplification of cDNA ends (RACE) (see, for example, Frohman et al., Proc. Nat. Acad. Sci. USA 85, 8998-9002, 1988). Modifications of the technique, exemplified by the Marathon technology (Clontech Laboratories Inc.) for example, have significantly simplified the search for longer cDNAs. In the Marathon technology, cDNAs have been prepared from mRNA extracted from a chosen tissue and an ‘adaptor’ sequence ligated onto each end. Nucleic acid amplification (PCR) is then carried out to amplify the “missing” 5′ end of the cDNA using a combination of gene-specific and adaptor-specific oligonucleotide primers. The PCR reaction is then repeated using ‘nested’ primers, that is, primers designed to anneal within the amplified product (typically an adapter specific primer that anneals further 3′ in the adaptor sequence and a gene specific primer that anneals further 5′ in the known gene sequence). The products of this reaction can then be analyzed by DNA sequencing and a full-length cDNA constructed either by joining the product directly to the existing cDNA to give a complete sequence, or carrying out a separate full-length PCR using the new sequence information for the design of the 5′ primer.

Recombinant polypeptides of the present invention may be prepared by processes well known in the art from genetically engineered host cells comprising expression systems. Accordingly, in a further aspect, the present invention relates to expression systems comprising a polynucleotide or polynucleotides, to host cells which are genetically engineered with such expression systems and to the production of polypeptides of the invention by recombinant techniques. Cell-free translation systems can also be employed to produce such proteins using RNAs derived from relevant DNA constructs.

For recombinant production, host cells can be genetically engineered to incorporate expression systems or portions of polynucleotides thereof. Polynucleotides may be introduced into host cells by methods described in many standard laboratory manuals, such as Davis et al., Basic Methods in Molecular Biology (1986) and Sambrook et al., 1989. Preferred methods of introducing polynucleotides into host cells include, for instance, calcium phosphate transfection, DEAE-dextran mediated transfection, transfection, micro-injection, cationic lipid-mediated transfection, electroporation, transduction, scrape loading, ballistic introduction or infection.

Representative examples of appropriate hosts include, e.g., bacterial cells, such as Streptococci, Staphylococci, E. coli, Streptomyces and Bacillus subtilis cells; fungal cells, such as yeast cells and Aspergillus cells; insect cells such as Drosophila S2 and Spodoptera Sf9 cells; animal cells such as CHO, COS, HeLa, C127, 3T3, BHK, HEK 293 and Bowes melanoma cells; and plant cells.

As understood in the art, a great variety of expression systems can be used, for instance, chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression systems may contain control regions that regulate as well as engender expression. Generally, any system or vector that is able to maintain, propagate or express a polynucleotide to produce a polypeptide in a host may be used. The appropriate polynucleotide sequence may be inserted into an expression system by any of a variety of well-known and routine techniques, such as, for example, those set forth in Sambrook et al., 1989. Appropriate secretion signals may be incorporated into the desired polypeptide to allow secretion of the translated protein into the lumen of the endoplasmic reticulum, the periplasmic space or the extracellular environment. These signals may be endogenous to the polypeptide or they may be heterologous signals.

Polypeptide portions of IGFBP-4 are contemplated for use in the methods of the present invention. In these embodiments, the portion of the gene product is an active portion having angiogenesis, metastasis or tumor development-inhibiting properties, or it has the ability to exert a beneficial effect on angiogenesis-dependent conditions. IGFBP-4 has been shown to be proteolyzed (see, e.g., Overgaard, J. Biol. Chem. 2000, 275(40):31128-33). It has been reported in the literature that a number of proteins that inhibit angiogenesis, including angiostatin, endostatin, pexstatin, tumstatin, laminin, and fibronectin, have increased anti-angiogenic activity when present in cleaved forms as compared to full-length forms. The resulting cleavage products possess anti-angiogenic activity. For example, the angiogenesis inhibitor, angiostatin, is derived from plasminogen, and the prothrombin kringle-2 domain is a cleavage product of prothrombin (Lee, et al., J. Biol. Chem. 1998, 273 (44):28805-12; Soff, G. A., Cancer Metastasis Rev. 2000, 19(1-2):97-107). A short peptide from matrix metalloproteinase-2 (P-2) has also been found to inhibit angiogenesis and tumor growth (U.S. Pub. No. 2002/0182215 A1, incorporated herein by reference in its entirety). Therefore, identified polypeptides, as well as naturally-occurring cleavage products, are contemplated for use according to the methods of the invention. The use of cryptic regions of ECM components having anti-angiogenic function are discussed in, e.g., Schenk, S., et al., Trends in Cell Biol. 2003, 13: 366-375 and Kalluri, R. Nat. Rev. Cancer 2003, 3: 422-433.

Proteins, recombinant or synthetic, can be purified to substantial purity by standard techniques well known in the art, including detergent solubilization, selective precipitation with such substances as ammonium sulfate, column chromatography, immunopurification methods, and others. See, for instance, R. Scopes, Protein Purification: Principles and Practice, Springer-Verlag: New York (1982); Deutscher, Guide to Protein Purification, Academic Press (1990). The protein may then be isolated from cells expressing the protein and further purified by standard protein chemistry techniques

The term “substantially purified” refers to IGFBP-4 that is substantially or essentially free of components that normally accompany or interact with the protein as found in its naturally occurring environment, i.e. a native cell, or host cell in the case of recombinantly produced IGFBP-4. When the IGFBP or variant thereof is recombinantly produced, the culture medium may represent less than about 30%, less than about 25%, less than about 20%, less than about 15%, less than about 10%, less than about 5%, less than about 4%, less than about 3%, less than about 2%, or less than about 1% (by dry weight) of chemical precursors or non-protein-of-interest chemicals. Thus, “substantially purified” IGFBP as produced by the methods of the present invention may have a purity level of at least about 30%, at least about 35%, at least about 40%, at least about 45%, at least about 50%, at least about 55%, at least about 60%, at least about 65%, at least about 70%, specifically, a purity level of at least about 75%, 80%, 85%, and more specifically, a purity level of at least about 90% or greater as determined by appropriate methods such as SDS/PAGE analysis, RP-HPLC, SEC, capillary electrophoresis inter alia.

VI. Methods for Patient Administration

The dosage ranges for the administration of IGFBP-4, or fragment thereof, depend upon the form of the gene product, and its potency, and are amounts large enough to produce the desired effect wherein angiogenesis, tumor metastasis, tumor growth, cell adhesion or cell migration are inhibited or otherwise altered in a way that is favorable for amelioration of disease. The dosage should not be so large as to cause adverse side effects, such as hyperviscosity syndromes, pulmonary edema, congestive heart failure, and the like. Generally, the dosage will vary with the age, condition, sex and extent of the disease in the patient and can be determined by one of skill in the art. The dosage can also be adjusted by the individual physician in the event of any complication.

A therapeutically effective amount is an amount of the IGFBP-4 protein or polypeptide, e.g., a portion of the gene product having angiogenesis-, tumor metastasis-, tumor growth-, cell adhesion- or cell migration-inhibiting properties, or effectiveness in treatment of an angiogenesis-dependent condition, sufficient to produce a measurable inhibition of angiogenesis, tumor metastasis, tumor growth, cell adhesion, cell migration or a measurable effect on an angiogenesis-dependent condition, in the tissue being treated. Inhibition of these symptoms can be measured according to methods described herein, or by other methods known to one skilled in the art. Methods for assessing effect on an angiogenesis-dependent condition will depend on the condition being treated. For the particular condition such methods will be known to those of skill in the art.

It is to be appreciated that the potency, and therefore an expression of a “therapeutically effective” amount can vary. However, as shown by the present assay methods, one skilled in the art can readily assess the potency of a gene product of this invention. Potency can be measured by a variety of means, including, but not limited to: the measurement of inhibition of angiogenesis in the CAM assay, in the in vivo rabbit eye assay, or in the in vivo chimeric mouse:human assay; the inhibition of tumor metastasis in the chick embryo model or in the murine model; the inhibition of cell adhesion in a cell adhesion assay; the inhibition of cell migration in a cell migration assay; or the inhibition of tumor growth in the chick embryo assay or the SCID mouse assay, all as described herein and in the literature and known to those of skill in the art, and the like assays.

A “therapeutically effective” amount of IGFBP-4 can be determined by prevention or amelioration of adverse conditions or symptoms of diseases, injuries or disorders being treated. For all the indications of use of IGFBP-4, the appropriate dosage will of course vary depending upon, for example, the tumor type and stage and severity of the disease disorder to be treated and the mode of administration. For example, tumor inhibition as a single agent may be achieved at a daily dosages from about to 0.1 mg/kg to 40 mg/kg body weight, preferably from about 0.2 mg/kg to about 20 mg/kg body weight of a binding protein of the invention. In larger mammals, for example, humans, as indicated daily dosage is from about 0.25 to about 5 mg/kg/day or about 70 mg per day for an average adult at a dose of 1 mg/kg/day conveniently administered parenterally, for example once a day. Dosage ranges for IGFBP-3 are described in U.S. Publication No. 20040127411, incorporated herein by reference.

The proteins or polypeptides of the invention can be administered parenterally by injection or by gradual infusion over time. Although the tissue to be treated can typically be accessed in the body by systemic administration and therefore most often treated by intravenous administration of therapeutic compositions, other tissues and delivery means are contemplated where there is a likelihood that the tissue targeted contains the target molecule. Thus, proteins or polypeptides of the invention can be administered intravenously, intraperitoneally, intramuscularly, subcutaneously, intracavity, transdermally, and can be delivered by peristaltic means.

Therapeutic compositions are conventionally administered intravenously, as by injection of a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. Precise amounts of active ingredient required to be administered depend on the judgment of the practitioner and are peculiar to each individual. However, suitable dosage ranges for systemic application are disclosed herein and depend on the route of administration. Suitable regimes for administration are also variable, but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

The present invention contemplates therapeutic compositions useful for practicing the therapeutic methods described herein. Therapeutic compositions of the present invention contain a physiologically tolerable carrier together with the protein or polypeptide as described herein, dissolved or dispersed therein as an active ingredient. In a preferred embodiment, the therapeutic protein or polypeptide composition is not immunogenic when administered to a mammal or human patient for therapeutic purposes.

As used herein, the terms “pharmaceutically acceptable,” “physiologically tolerable,” and grammatical variations thereof, as they refer to, e.g., compositions, carriers, diluents, reagents and excipients, are used interchangeably and represent that the materials are capable of administration to or upon a mammal without the production of undesirable physiological effects such as nausea, dizziness, gastric upset and the like.

The preparation of a pharmacological composition that contains active ingredients dissolved or dispersed therein is well understood in the art and need not be limited based on formulation. Typically such compositions are prepared as injectables either as liquid solutions or suspensions, however, solid forms suitable for solution, or suspensions in liquid prior to use can also be prepared. The preparation can also be emulsified.

The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic, etc. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes.

Liquid compositions can also contain liquid phases in addition to and to the exclusion of water. Exemplary of such additional liquid phases are glycerin, vegetable oils such as cottonseed oil, and water-oil emulsions.

In further embodiments, the invention enables any of the foregoing methods to be carried out in combination with other therapies such as, for example, treatment with another compound, e.g., an inhibitor of angiogenesis and tumor development processes (e.g., a monoclonal antibody that binds to the cryptic collagen epitope, HUIV26), chemotherapy or radiation therapy, or treatment with cytotoxic agents. Chemotherapeutic agents useful in the methods of the present invention include, e.g., taxanes (i.e., Taxol, Docetaxel, Paclitaxel), dacarbazine (DTIC), Adriamycin, Bleomycin, Gemcitabine, Cyclophosphamide, Oxaliplatin, Camptothecan, Ironotecan, Fludarabine, Cisplatin and Carboplatin.

An angiogenesis inhibitor may be administered to a patient in need of such treatment before, during, or after chemotherapy. It is also preferred to administer an angiogenesis inhibitor to a patient as a prophylaxis against metastases after surgery on the patient for the removal of solid tumors

VII. Cell Types

The methods of the present invention can be practiced using a number of cell lines which are obtained and maintained according to methods known to those of skill in the art. For example, murine B16F10 melanoma cell line was obtained from ATCC (Rockville, Md.). Tumor cells were maintained in Dulbecco's Modified Eagles Medium (DMEM) (Gibco Grand Island N.Y.) supplemented with 10% Fetal Bovine Serum (FBS) (Hyclone, Logan Utah), 1.0% Sodium Pyruvate, Glutamate and Pen-Strep (Gibco, Grand Island N.Y.). Cells were maintained as sub-confluent cultures before use and harvested with trypsin-EDTA (Gibco, Grand Island N.Y.).

Cell lines described herein have been previously described, as follows: ECV and ECVL in Brooks, et al., Cell 1998, 92:391-400; M21 and M21L in Montgomery, et al., Proc. Natl. Acad. Sci. USA 1994, 91:8856-8860, and; CS1 and b3CS1 in Brooks, et al., Cell 1996, 85:683-693.

The patents and publications cited herein reflect the level of skill in this field and are hereby incorporated by reference in their entirety to the same extent as if each was specifically and individually indicated to be incorporated by reference.

### EXAMPLES

The present invention is further illustrated by the following examples, which should not be construed as limiting in any way.

### Example I

### Inhibition of Cellular Interactions with the HUIV26 Cryptic Collagen Epitope Enhances Expression of IGFBP-4 RNA

Differential cDNA array analysis suggested increased expression of IGFBP-4 RNA in tumor cells treated with Mab HUIV26.

An Affymetrix™-based differential cDNA array analysis was performed using B16F10 tumor cells treated or not treated with Mab HUIV26. Non-tissue culture treated dishes were coated overnight with 100 μg/ml of denatured collagen IV in PBS. The next morning the plates were washed and incubated in blocking solution (1% BSA in PBS) for approximately 30 minutes.

Tumor cells (7×10⁶) were resuspended in serum-free media and added to each plate in the presence or absence of Mab HUIV26 or a control isotype-matched IgM antibody (100 μg/ml). The cells were allowed to incubate for a total of 12 hours. Following the 12-hour incubation period, the cells were harvested and the RNA was isolated using both a TRIzol reagent and the Qiagen Rneasy Mini Protocol for RNA Cleanup. After RNA extraction, the amount and quality of RNA was quantified utilizing a spectrophotomer, and 5-8 μg of total RNA was utilized to synthesize double-stranded cDNA.

The first cDNA strand was obtained using a reaction mixture containing a T7-(dT)24 Primer, 1× First Strand Buffer, 0.1M DTT and 10 mM dNTP mix in addition to the extracted RNA. The tubes were incubated at 42° C. for approximately 1.5 hours.

For the second strand cDNA synthesis, a 1× Second Strand Buffer, 10 mM dNTP mix, 10 U/ml of E. coli DNA Ligase, 10 U/ml of DNA Polymerase I and RNaseH were added and allowed to incubate at 16° C. for 2.5 hours. Following the incubation period, T4 DNA Polymerase was added and the tubes were incubated for 5 minutes and stored at −80° C. The final double-stranded cDNA product was cleaned utilizing phenol extraction and ethanol precipitation. Next, the synthesized cDNA was converted to cRNA and labeled with biotin labeled ribonucleotides in a reaction mixture that also included HY Reaction Buffer, 10×DTT, Rnase Inhibitor Mix and 20×RNA Polymerase. The final cRNA product was cleaned utilizing the Qiagen Rneasy Mini Protocol for RNA Cleanup and 15 μg of cRNA was fragmented and hybridized to a U95Av2 chip.

Analysis of the RNA showed increased expression of IGFBP-4 in cells treated with Mab HUIV26.

Relative expression levels of IGFBP-4 were assessed by both real time RT-PCR and Western Blot analysis. Tumor cells (B16F10) were allowed to interact with denatured collagen type-IV in the presence or absence of Mab HUIV26 or an isotype-matched control antibody, and mRNA and whole cell lysates were prepared.

Real Time quantitative RT-PCR was carried out essentially as previously described with some modifications (Livak et al., Method 2001, 25:402-408). Total RNA was isolated using RNeasy miniprep columns (Qiagen, Valencia Calif.) according to the manufacturer's instructions. Total RNA (1 μg) was reverse transcribed using 1× Reverse Transcriptase Buffer, MgCl₂ (3 mM), dNTP (2.0 mM), RNAse inhibitor (0.2 U/μl), random hexamer primers (0.5 mM), and MMLV reverse transcriptase (0.3 U/μl) in 20 μl reactions using a 3-step cycle (Promega, Madison, Wis.). Real-time fluorescence detection was carried out using an ABI Prism 7900 Sequence Detection System. Reactions were carried out in microAmp 96 well reaction plates. Primers and probes were designed using Primer 3 version 2 and ENSEMBL software (Promega).

The following human-specific real time PCR primer pairs were used to detect IGFBP-4:

| | |
|---|---|
| 5′-CCTGCACACACTGATGCAC-3′ | (SEQ ID NO: 1) |
| and | |
| 5′-GTCTCGAATTTTGGCGAAGT-3′. | (SEQ ID NO: 2) |

The primers used to detect control gene β2-macroglobulin were:

(forward; SEQ ID NO: 3)

5′-AAAGATGAGTATGCCTGCCG-3′
and

(reverse; SEQ ID NO: 4)

5′-CCTCCATGATGATGCTGCTTACA-3′.

cDNA from samples were labeled with SYBR Green (Roche) and real time PCR was run using a Light Cycler (NYU Genomic Core Services). Quantification of data was performed using Light Cycler real time PCR analysis software package 3.5 (Roche).

Fold induction was calculated using methods described by Livak et al., 2001. Amplification products utilized through Sybergreen detection were initially checked by electrophoresis on ethidium bromide stained agarose gels. The estimated size of the amplified products matched the calculated size for transcript by visual inspection.

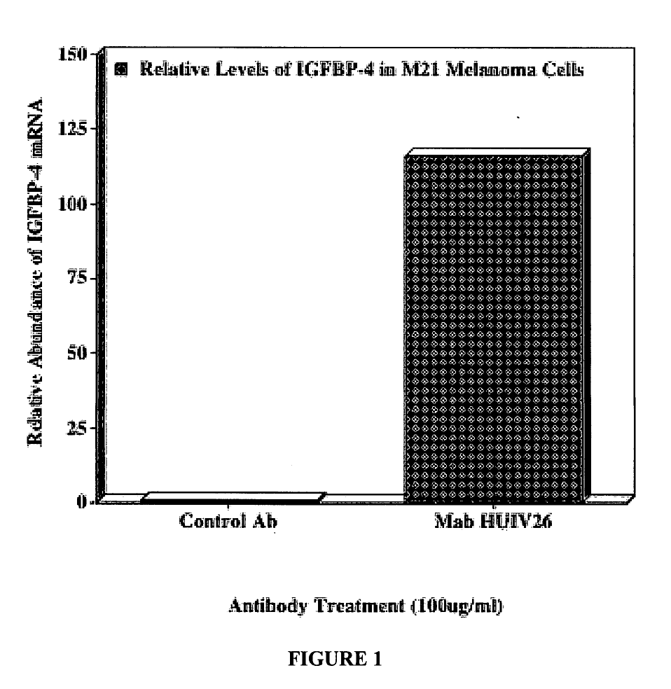

As shown in FIG. 1, incubation of cells with Mab HUIV26 resulted in an approximately 115-fold increase in the relative levels of IGFBP-4 RNA as compared to isotype-matched controls. These findings are further evidence that blocking cellular interactions with a αvβ3 ligand (HUIV26 cryptic epitope) enhances expression of endogenous angiogenesis inhibitors.

### Example II

### Isolation of αvβ3 Expression Variants of Human ECV304 Bladder Carcinoma Cells

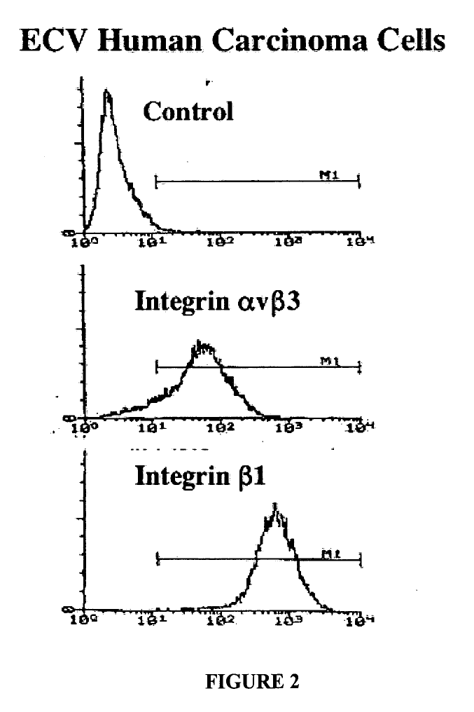

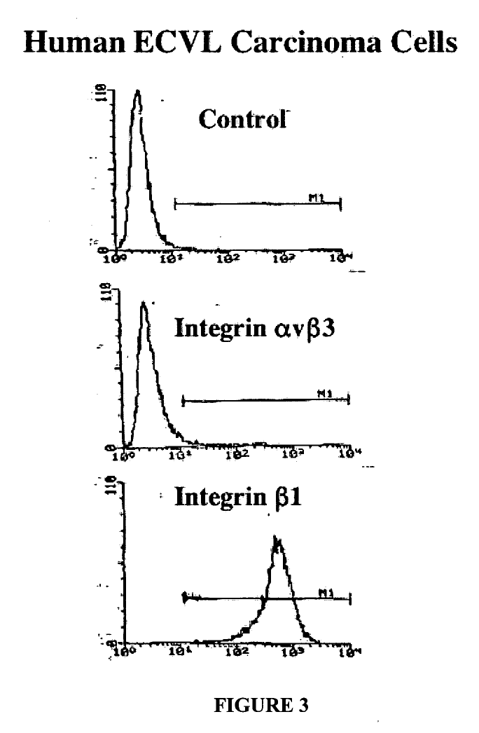

To examine the functional significance of αvβ3 on tumor growth in a histologically distinct tumor type, we isolated variants of the human bladder carcinoma cell line ECV304 that either expressed (ECV) or lacked expression (ECVL) of αvβ3. To isolate these variants, ECV cells we subjected to Fluorescence Activated Cell Sorting (FACS) of cells stained with Mab LM609 directed to αvβ3 integrin. Briefly, ECV cells were incubated with Mab LM609 and FACS sorted. ECV cells that failed to express cell surface αvβ3 were expanded. The negative FACS selection procedure was carried out a total of 4 times to ensure a stable population of αvβ3 negative ECV cells. As shown in FIG. 2, the parent ECV carcinoma cells expressed high surface levels of αvβ3 (middle panel) and β1 integrins (bottom panel). In contrast, negatively-selected (ECVL) cells (FIG. 3) expressed no detectable αvβ3 on the cell surface (middle panel). Reduction of αvβ3 expression in these cells resulted in little if any change in β1 integrin expression (bottom panel).

### Example III

### Elevated Levels of IGFBP-4 Protein in Conditioned Medium from Tumor Cells Lacking αvβ3

Differential cDNA array analysis suggested increased expression of IFGBP-4 in ECVL as compared to ECV cells. The Affymetrix™-based differential cDNA array analysis was performed similarly to that described in Example I, comparing ECV and ECVL cells.

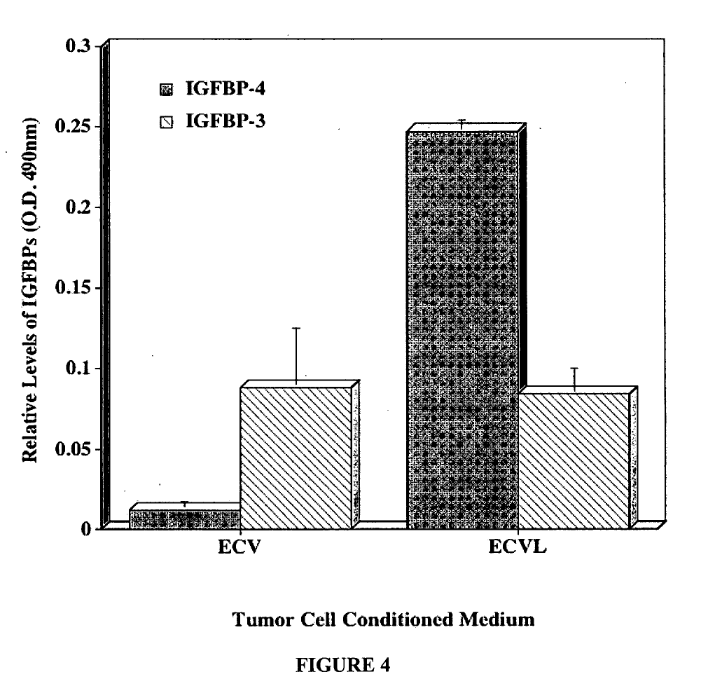

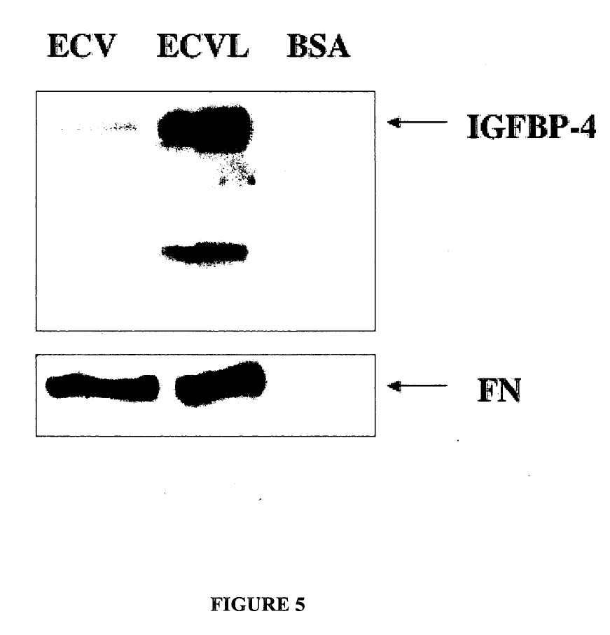

The relative levels of IGFBP-4 were analyzed in conditioned medium (CM) from ECV and ECVL cells by solid phase ELISA (FIG. 4) and Western blot (FIG. 5). As shown by ELISA, the relative levels of IGFBP-4 increased in CM from ECVL by greater than 10-fold as compared to ECV, while little if any change in the levels of IGFBP-3 was observed. To confirm these findings, Western blot analysis was carried out. As shown in FIG. 5, IGFBP-4 was dramatically increased in the CM of ECVL cells as compared to ECV cells while little if any change was detected in soluble fibronectin.

### Example IV

### Recombinant Human IGFBP-4 Inhibits bFGF-Induced Angiogenesis In Vivo

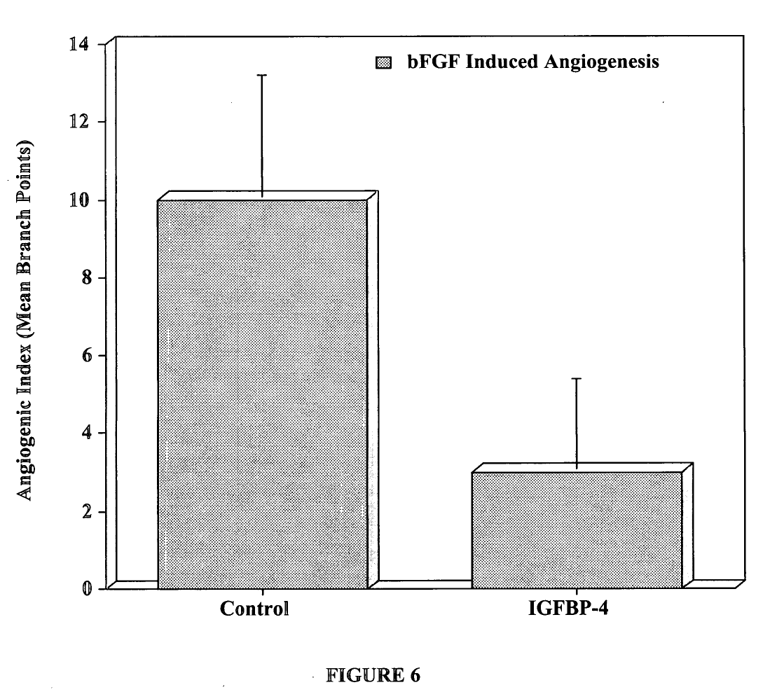

To examine the effects of recombinant IGFBP-4 on bFGF-induced angiogenesis, angiogenesis was measured in the chick CAM assay, described herein. Angiogenesis was induced in the CAMs of 10-day old chick embryos. Twenty-four hours later, the embryos were treated topically with control BSA or recombinant IGFBP-4 (100 ng/embryo). CAMs were removed and angiogenesis quantified. As shown in FIG. 6, IGFBP-4 (100 ng) significantly (P<0.001) inhibited bFGF-induced angiogenesis by greater than 70% as compared to control. These results suggest that IGFBP-4 represents a new endogenously expressed inhibitor of angiogenesis.

### Example V

### Recombinant IGFBP-4 Inhibits M21 Cell Adhesion to Denatured Collagen-IV

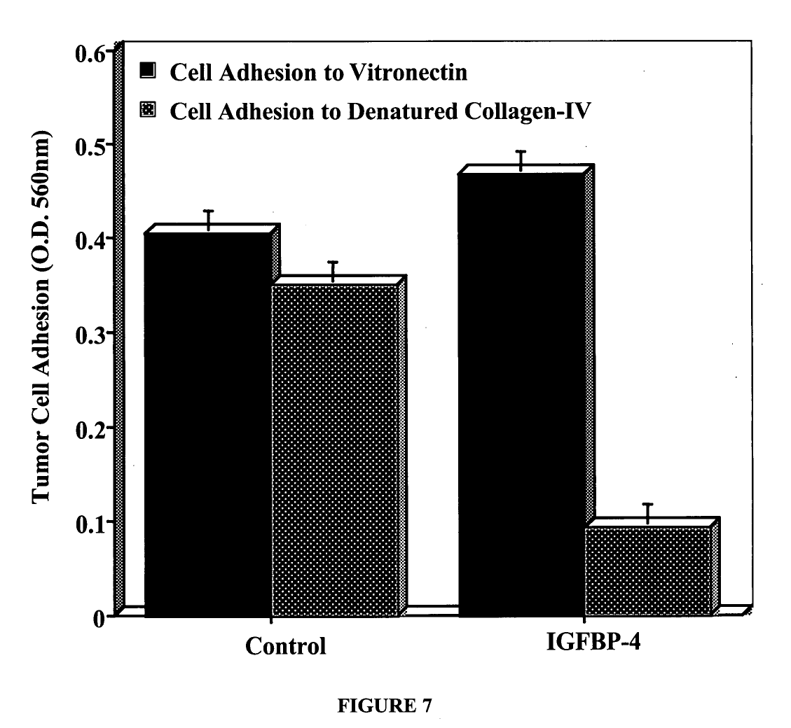

To examine whether IGFBP-4 could modulate cell adhesion, in vitro assays were performed. Briefly, culture plates were coated with either vitronectin or denatured collagen type-IV. M21 cells were incubated for 1 hour in the presence or absence of recombinant IGFBP-4 (200 ng/ml) or control BSA. Cells were allowed to attach for 20 minutes and non-attached cells removed by washing. Cell adhesion was quantified by measuring the O.D. of cell associated eluted dye as described (Xu, et al., J. Cell Biol. 2001, 154: 1069-1079). As shown in FIG. 7, IGFBP-4 potently inhibited M21 cell adhesion to denatured collagen type-IV by approximately 70% while exhibiting little effect on adhesion to vitronectin. These data suggest that IGFBP-4 may inhibit cell adhesion in a ligand and/or integrin specific manner.

### Example VI

### Inhibition of αvβ3 Ligation Upregulates IGFBP-4

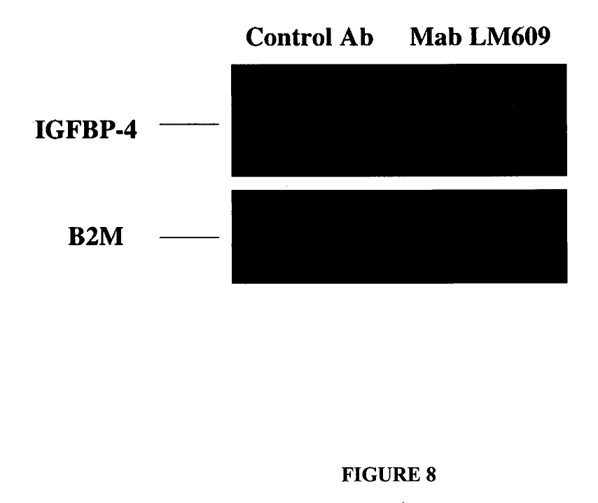

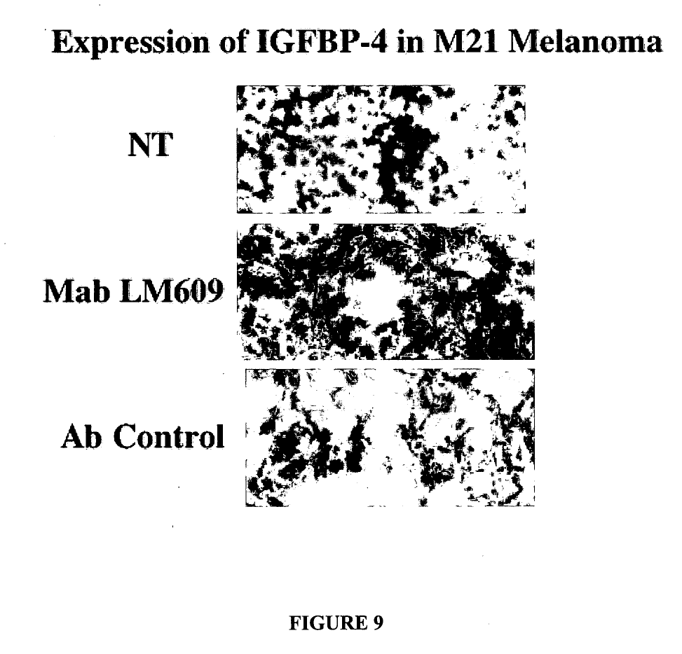

The effects of blocking αvβ3-mediated interactions on IGFBP-4 expression were examined by treating cells interacting with the known αvβ3 ligand, denatured collagen type-IV, with the anti-αvβ3 monoclonal antibody LM609 (described previously, e.g. in U.S. Publication No. 2005/0002936). M21 cells were allowed to interact with denatured collagen type-IV in the presence or absence of Mab LM609 or an isotype matched control antibody for 12 hours, and levels of IGFBP-4 RNA were measured by PCR. As shown in FIGS. 8 and 9, expression of IGFBP-4 was significantly enhanced in M21 cells and M21 tumors grown in the chick embryo following treatment with Mab LM609. These data suggest that specific inhibition of αvβ3 may increase expression of IGFBP-4 in vitro and in vivo.

### Example VII

### Treatment of Established MCF-7 Human Breast Tumors with rhIGFBP-3 and Paclitaxel

Female Balb/c mice (8 animals per group) receive bilateral subcutaneous implants of MCF-7 breast tumor fragments which are allowed to grow to volumes of 100-150 mm³ prior to initiation of treatment. Upon establishment of the tumors, mice are treated with either IGFBP-4 (3, 10 or 30 mg/kg twice daily, subcutaneously for 21 days), paclitaxel (10 or 20 mg/kg, once daily, intraperitoneally for 5 days) or a combination of agents. Tumors are measured twice weekly for 3 weeks and net tumor growth is calculated at each time point. Results indicate reduced tumor growth in the mice treated with IGFBP-4, and further reduction in those treated with the combination therapy.

### Example VIII

### Treatment of a Patient with Metastatic Breast Cancer

A patient with breast cancer metastatic to the liver has blood drawn for liver function tests. The patient undergoes an abdominal CT scan in order to note the size and number of the liver metastases. The patient's overall medical condition is assessed by a health professional using physical examination; blood tests such as a complete blood count, BUN, and creatinine; and EKG.

An IGFBP-4 dose based on a total dose of 1 mg/kg/day is mixed in aqueous solution and administered intravenously through a peripheral vein catheter over a two-hour period. Following infusion of IGFBP-4, the patient is monitored for two hours by a health professional for the appearance of adverse effects. In the absence of such effects, the patient is discharged home.

Two weeks following IGFBP-4 infusion, the patient has repeat liver function tests and CT scan. Lowering of the liver function test values may be indicative of tumor metastases regression. CT scan visualization of decreased size and/or number of metastases is indicative of successful treatment of the metastases.

What is claimed is:

1. A therapeutic composition comprising IGFBP-4 and a pharmaceutically acceptable excipient.

2. A method for inhibiting angiogenesis in a patient, treating a tumor in a patient, inhibiting metastases in a patient, or treating an angiogenesis-dependent condition in a patient, comprising: administering a therapeutically effective amount of IGFBP-4 to the patient.

3. The method of claim 2 wherein the IGFBP-4 is administered: intravenously, intraperitoneally, intramuscularly, subcutaneously, intracavity, transdermally, topically, intraocularly, orally, intranasally, or by peristaltic means.

4. The method of claim 2 wherein the IGFBP-4 is administered in combination with a chemotherapeutic agent, a radioactive material, or a cytostatic agent.

5. The method of claim 2 wherein the patient is a mammal.

6. The method of claim 5 wherein the patient is a human.


SEQUENCE LISTING (4 sequences)

SEQ ID NO: 1 - DNA, 19 bp, Artificial
  PCR primer
cctgcacacactgatgcac

SEQ ID NO: 2 - DNA, 20 bp, Artificial
  PCR primer
gtctcgaattttggcgaagt

SEQ ID NO: 3 - DNA, 20 bp, Artificial
  PCR primer
aaagatgagtatgcctgccg

SEQ ID NO: 4 - DNA, 23 bp, Artificial
  PCR primer
cctccatgatgatgctgcttaca